(19) United States
(12) Patent Application Publication
(10) Pub. No.: US 2023/0365570 A1
(43) Pub. Date: Nov. 16, 2023

(54) COMPOUNDS WITH GLUCOCORTICOID SPARING EFFECTS AND USES THEREOF

(71) Applicant: The Regents of the University of California, Oakland, CA (US)

(72) Inventors: Dennis A. Carson, La Jolla, CA (US); Tomoko Hayashi, San Diego, CA (US); Mary Patricia Corr, San Diego, CA (US); Howard B. cottam, Escondido, CA (US); Tadashi Hosoya, Bunkyo-ku (JP); Nikunj Shukla, San Diego, CA (US)

(21) Appl. No.: 18/029,899

(22) PCT Filed: Oct. 5, 2021
(86) PCT No.: PCT/US2021/053648

§ 371 Date: Mar. 31, 2023

Related U.S. Application Data
(60) Provisional application No. 63/087,660, filed on Oct. 5, 2020.

(51) Int. Cl. C07D 487/04; A61P 37/06; C07D 307/72

(52) U.S. Cl. CPC C07D 487/04; A61P 37/06; C07D 307/72

(57) ABSTRACT

The disclosure relates to 1H-pyrazolo[3,4-d]pyrimidin-4-amine compounds and 1-nitro-5-amido-disubstituted furan compounds, or a pharmaceutically acceptable salt thereof, and methods for their use in treating, among other conditions, inflammatory diseases or an autoimmune diseases.

### CROSS-REFERENCE TO RELATED APPLICATIONS

This application claims the benefit of U.S. Provisional Appl. Ser. No. 63/087,660, filed Oct. 5, 2020, which is incorporated by reference as if fully set forth herein.

### STATEMENT OF U.S. GOVERNMENT SUPPORT

This invention was made with government support under grant HHSN272201400051C awarded by National Institutes of Health. The government has certain rights in the invention.

### BACKGROUND

The development of immunosuppressive drugs and target-based drugs has expanded the therapeutic armamentarium in the treatment of inflammatory and autoimmune diseases. Despite these advances, glucocorticoids (GCs) remain the most reliable agents as an initial treatment in the acute phase of the disease and the maintenance therapy for preventing disease relapse GCs are a double-edged sword because long-term use can induce adverse events, including cardiovascular disease, osteoporosis, cataracts and muscle atrophy, in addition to the risk of serious infections. To minimize the adverse events of GCs, the American College of Rheumatology (ACR) and European League Against Rheumatism (EULAR) established guidelines regarding the clinical use of GCs in rheumatic diseases. To reduce side effects several clinical trials examined whether the use of the conventional immunosuppressive drugs could induce remission without oral GC use, Combined these studies reported that the management of rheumatic diseases still relies on GCs as part of the therapeutic regimen with its relatively non-specific, but strong anti-inflammatory effects. Hence there remains a clinical need for drugs which can dose-spare or replace the anti-inflammatory effects of GCs.

One of the key mechanisms for the anti-inflammatory effect of GCs is the regulation of nuclear factor kappa B (NF-κB) through IKKß. NF-κB is an essential transcription factor induced by inflammatory responses and plays critical roles in cell cycle progression, cell survival, adhesion, and inhibition of apoptosis. Several human genetic diseases confirm the multifunction of NF-κB including genetic defects in NF-κB activating molecules (e.g., NEMO) resulting in an immunodeficiency phenotype and in NF-κB regulatory molecules, which causes an autoinflammatory phenotype. When NF-κB is activated, its activation is transient and regulated by the consumption of downstream adaptor molecules and the induction of anti-inflammatory molecules. In chronic inflammatory diseases, such as rheumatic diseases, autoinflammatory diseases, and inflammatory bowel diseases, excessive and continuous activation of NF-κB are common findings reflecting a large amount of inflammatory stimuli and the dysregulation of negative-feedback mechanisms GC's anti-inflammatory mechanisms were attributed to inhibitory effects against NF-κB by interfering with DNA binding competitively and inducing anti-inflammatory genes. Also, several immunosuppressants and disease-modifying anti-rheumatic drugs (DMARDs), such as calcineurin inhibitor, iguratimod, and methotrexate, attenuated NF-κB activity at least indirectly. These findings indicate that at least partial inhibition of NF-κB signaling pathway remains a promising therapeutic strategy. However, despite the intensive effort to discover and develop NF-κB targeting drugs, few agents have been approved for clinical use because of unexpected adverse events, including nephrotoxicity, neuropathy, and paradoxical IL-1ß release.

### BRIEF DESCRIPTION OF THE FIGURES

The drawings illustrate generally, by way of example, but not by way of limitation, various embodiments discussed herein.

FIG. 1A is a workflow strategy for identifying NF-κB suppressive compounds by mining existing HTS data and subsequent screening strategy.

FIG. 1B is a distribution plot of NF-κB reporter activity from compounds screened in two prior HTS relative to known bioactive compounds. Two prior HTS were conductedusing the same cell-based FRET assay with THP-1 CellSensor NF-κB reporter cells. The first tested the activation/inhibition of reporter signal with compound treatment at 5 h (HTS1, x-axis), and other tested the reporter signal with LPS plus compound at 12 h (HTS2, y-axis), using 155,452, and 165,099 compound libraries, respectively. Results of the 134,115 overlapping compounds were normalized to the LPS controls in each assay and are plotted individually. Compounds in an area (black box) encapsulating known GC and below the activity of gonadal steroids or COX inhibitors (X axis −10%-0% and Y axis <30%) were picked for further assessment of immunosuppressant activity.

FIGS. 2A-2F are data from a confirmation screen of compounds for NF-κB reporter activity, viability and chemokine stimulation; (A-C): Confirmation screening of initial 1824 hit compounds. Inhibition of an LPS induced signal by compound as a percent NF-κB activity relative to LPS controls at 5 h; (A), 16 h; (B) and both; (C). 1824 compounds were assessed with LPS as the primary stimulus using the FRET assay with THP-1 CellSensor NF-κB reporter cells. The percent NF-κB activation was calculated relative to DMSO LPS (red) as 100% and DMSO as vehicle control (magenta) as 0%. All assay runs included DEX+LPS (green), and UTC LPS (brown) as positive controls. Gray dots represent the duplicate data of each of the tested compounds. 122 compounds were selected based on the “Top-X” criteria: <25% at 16 h or <50% at 5 h (dotted lines) (D and E), Relative CXCL8 production and cellular viability of THP-1 cells treated with the 122 selected compounds. THP-1 cells treated with LPS (10 ng/ml) and 122 hit compounds (5 iM) were analyzed for CXCL8 production by ELISA; (D), and tested for cellular toxicity by MTT assay; (E). Results were normalized relative to the LPS controls, and ranked by CXCL8 production. The compounds in (D) and (E) are presented in the same order. (F) Both assays are plotted and the dotted line indicates 70% production of CXCL8 and 90% viability relative to the control. DEX (blue) and UTC (brown) were used as positive controls. Data shown as mean ±SD of triplicate data.

FIGS. 3A-3D are results from Cytokine and chemokine suppression by the lead compounds in THP-1 cells (A-C) CXCL8 production by THP-1 cells treated with graded concentrations of the indicated compounds and stimulated with 10 ng/ml of LPS; (A), 2 ng/ml of TNF; (B), or 2 ng/ml of IL-1ß; (C) overnight; (0) TNF production from THP-1 cells stimulated with 2 ng/ml of IL-1ß and compounds overnight. Candidate compounds were added at indicated concentrations with 0.1% final concentration of DMSO as vehicle. Shown are mean ±SEM and * indicates p<0.05, **p<0.01, ***p<0.001 & p<0.0001 by ANOVA with Dunnett's post hoc test comparing compound against vehicle +LPS, TNF or IL-11 respectively. Data are representative of two independent experiments showing similar results,

FIGS. 4A-4C are results showing the potency of lead compounds combined with DEX in inhibiting CXCL8 production. Compounds 1-1; (A), 1-2; (B) and 3-1; (C) were added at the indicated concentrations and DEX was added at 100 nM with 0.04% final concentration of DMSO as vehicle to TNF 2 ng/ml stimulated THP-1 cells overnight. CXCL8 was measured in the supernatant. Data are represented as mean ±SEM and indicates p<0.05, **p<0.01, ***p<0.001, & p<0.0001 by two way ANOVA with Bonferroni post hoc test comparing compound vs. compound DEX. Data are representative of two independent experiments showing similar results.

FIGS. 5A-5D are results showing synergistic effects of lead compounds with DEX in suppressing TNF stimulated CXCL8 production. Compounds 1-1; (A), 1-2; (B) and 3-1; (C) and DEX were titrated to the indicated concentrations and added to TNF 2 ng/ml stimulated THP-1 cells overnight and CXCL8 measured in the supernatant. Vehicle was 0.04% DMSO. Data are represented as mean ±SEM. (D) Potency ratios were calculated and presented as isobolograms. The dotted line represents additivity between DEX and the compounds. Data are representative of two independent experiments showing similar results.

FIGS. 6A-6F are results showing that compound 1-1 suppresses chemokine and cytokine release by TNF stimulated RA FLS; (A-E) Chemokine and cytokine production by RA FLS stimulated with 1 ng/ml of TNF for overnight with graded dilutions of compound 1-1, The supernatants were assayed for IL-6; (A), CXCL8; (B), MMP-3; (C), CXCL1; (0) and CCL2; (E). (F) Cell viability was assessed by WST-8 assay. DMSO was 0.1% of the final concentration as vehicle. Data are represented as mean ±SD. The 1050 values are shown, Data are representative of two independent experiments showing similar results.

FIGS. 7A-7D are results showing synergistic suppression of CXCL8 and 1L-6 production by compound 1-1 and DEX. Levels of CXCL8; (A) and 1L-6; (B) secreted into the supernatant by RA FLS stimulated overnight with 1 ngiml of TNF and treated with the indicated concentrations of 1-1 and DEX were measured by ELISA. Data are represented as mean ±SEM. Potency ratios were calculated and presented as isobolograms for CXCL8; (C) and IL-6; (D). The dotted line represents additivity between DEX and the compounds. Data are representative of two independent experiments showing similar results,

FIGS. 8A-8B show the (A) syntheses of select nitrofuranylamide analogs 4, 5, and 6 and (B) cytotoxicity as well as cytokine suppression by these compounds at 10 uM concentration induced by LPS in human THP-1 cells. *P<0.05, **P<0.01, ***P<0,001 by ANOVA with Dunnetts compared to vehicle.

FIGS. 9A-9F show the partial STING dependent activity of nitrofuranylamide compounds 4, 5 and 6 and inhibition of TLR pathways. (A-DO These compounds inhibited mTNF-a and mIP-10 production induced by TLR4 (LPS), TLR7 (1V270) and STING (DMXAA) ligands in BMDCs obtained from WT and STING knock-out Tmem173−/− mice. *P<0.01 by ANOVA with Dunnett's compared to Veh. (E) Dose-dependent inhibition of TNF-α induced by IL-1β in THP-1 cells and (F) IFNβ induced by DMXAA in rnBMDCs.

FIGS. 10A-10D show the in vivo activity of compound 5 in WT and STING knock-out Tmem173^{-/-} mice. Mice were induced with K/B×N serum on days 0 and 2 to have arthritis and related pain. Compound 5 was administered (750 nmol) IP BID on days 0-5. (A) Compound 5 has minimal effects on acute paw swelling but significantly reduces allodynia. (B) These effects of compound 5 are not seen in similarly treated Tmem173−/− mice. Mechanical allodynia is measured with monofilaments in a von Frey assay and increase in pain perception by the animal is recorded as a drop in the threshold. *P<0.0001 by two-way ANOVA.

### SUMMARY

By reanalyzing data from two prior high throughput screens (HTS) that utilized a NF-κB reporter construct in THP-1 cells, 1824 small molecule synthetic compounds were identified herein that demonstrated NF-κB suppressive activities similar to the glucocorticoids included in the original >134,000 compound libraries,

These 1824 compounds were then rescreened for attenuating NF-κB activity at 5 and 16 h after LPS stimuli in the NF-κB THP-1 reporter cells. After a “Top X” selection approach 122 hit compounds were further tested for toxicity and suppression of LPS induced CXCL8 release in THP-1 cells. Excluding cytotoxic compounds, the remaining active compounds were grouped into chemotype families using Tanimoto based clustering. Promising representatives from clustered chemotype groups were commercially purchased for further testing. Amongst these index compounds a lead chemotype: 1H-pyrazolo-[3,4 d]-pyrimidin-4-amine, effectively suppressed CXCL8, and TNF production by THP-1 cells when stimulated with LPS, TNF or IL-1ß. Extending these studies to primary cells, these lead compounds also reduced IL-6 and CXCL8 production by TNF stimulated fibroblast-like synoviocytes (FLS) from rheumatoid arthritis (RA) patients. Importantly a lead 1H-pyrazolo-[3,4 d]- pyrimidin-4-amine compound demonstrated synergistic effects with dexamethasone when co-administered to TNF stimulated THP-1 cells and RA FLS in suppressing chemokine production. In summary, a cell based HTS approach identified lead compounds that reduced NFκKB activity and chemokine secretion induced by potent immunologic stimuli, and one lead compound that acted synergistically with dexamethasone as an anti-inflammatory agent showing a dose-sparing effect.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure relates to compounds of the formula (I):

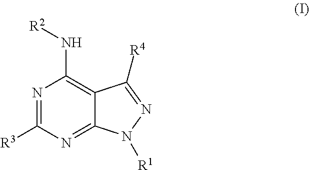

or pharmaceutically acceptable salt thereof,
wherein;
R¹ is aryl or heterocyclyl;
R² is alkyl or cycloalkyl, wherein the cycloalkyl is not amino- or amido-substituted when R¹ is aryl;
R³ and R⁴ are each interpedently H or alkyl; and
the compound is not a compound of the formula:

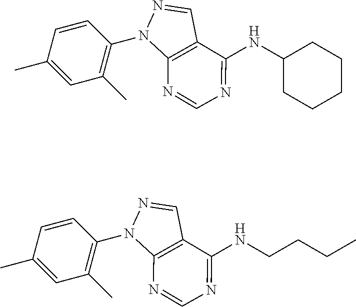 [SMILES: Cc1ccc(-n2ncc3c(NC4CCCCC4)ncnc32)c(C)c1 ; CCCCNc1ncnc2c1cnn2-c1ccc(C)cc1C]

The disclosure also relates to compounds of the formula (II):

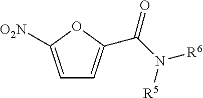

or pharmaceutically acceptable salt thereof,
wherein:
R⁵ is H, alkyl or OR⁷, wherein R⁷ is H or alkyl; and
R⁶ is aryl, monocyclic pyrrolidinyl or pyrrolyl; bicyclic furany; thiophenyl; and benzimidazolyl;
wherein R⁵ is alkyl or OR' when R⁶ is aryl, thiopehnyl, indolyl or benzimidazolyl. R⁶ can be a four-, five- or six-membered heterocyclyl group. Examples of heterocyclyl group include azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl or piperazinyl. Alternatively, R⁶ is aryl.

The disclosure also relates to a method for reducing at least one of NF-κB activity and chemokine secretion induced by immunologic stimuli, the method comprising administering at least one of a 1H-pyrazolo[3,4-d]pyrimidin-4-amine compound and an 1-nitro-5-amido-disubstituted furan, or a pharmaceutically acceptable salt thereof, to a subject in need thereof. The methods can further comprise administering a glucocorticosteroid or a pharmaceutically acceptable salt thereof. The 1H-pyrazolo[3,4-d] pyrimidin-4-amine compound can act synergistically with the glucocorticosteroid, causing a dose-sparing effect with regard to the glucocorticosteroid, such that less glucocorticoid needs to be used to obtain a therapeutic effect that could be achieved with higher glucocorticoid doses (for example, the compounds described herein can lower the dose of steroids needed to attain an anti-inflammatory effect). Examples of the at least one 1 H-1-pyrazolo[3,4-d] pyrimidin-4-amine is a compound of the formula:

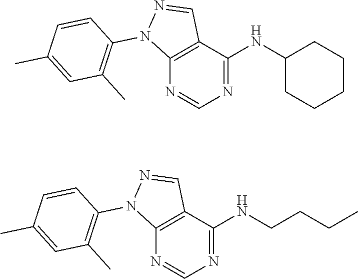 [SMILES: Cc1ccc(-n2ncc3c(NC4CCCCC4)ncnc32)c(C)c1 ; CCCCNc1ncnc2c1cnn2-c1ccc(C)cc1C]

a pharmaceutically acceptable salt thereof. Examples of the 1H-pyrazolo[3,4-d] pyrimidin-4-amines also include compounds of the formula (I):

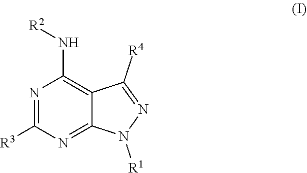

or pharmaceutically acceptable salt thereof,
wherein:
R¹ is aryl or heterocyclyl; and
R² is alkyl or cycloalkyl. R¹ can be heterocyclyl, such as a four-, five- or six-membered heterocyclyl group. Examples of heterocyclyl groups include azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrroiidinyl, piperidinyl or piperazinyl. Alternatively or in addition, R³ or R⁴ can be H. Or R³ or R⁴ can be alkyl. Alternatively, R¹ can be aryl substituted with alkyl or cycloalkyl. An example of alkyl is (C₁-C₆)-alkyl. In some examples, R¹ is not substituted with two CH3 groups, such as with two CH3 groups that are meta to one another. Alternatively or in addition, R² can be (C₁-C₁₀)-alkyl or (C₃-C₆)-cycloalkyl, such as (C₁-C₃)-alkul, (C₅-C₁₀)-cycloalkyl or (C₃-C₅)-cycloalkyl.

Examples of 1-nitro-5-amido-disubstituted furan that can be used in the methods described herein include compounds of the formula (II):

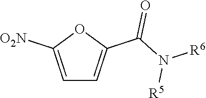

or pharmaceutically acceptable salt thereof,
wherein:
R⁵ is H, alkyl or OR⁷, wherein R⁷ is H or alkyl; and
R⁶ is aryl or heterocyclyl. R⁶ can be a four-, five- or six-membered heterocyclyl group. Examples of heterocyclyl group include azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl or piperazinyl. Alternatively, R⁶ is aryl.

The methods described herein also include a method for treating an inflammatory disease or an autoimmune disease comprising administering a therapeutically effective amount of at least one of a 1H-pyrazolo[3,4-d]pyrirnidin-4-amine compound and a 1-nitro-5-amido-disubstituted furan, or a pharmaceutically acceptable salt thereof, to a subject in need thereof.

This disclosure also contemplates pharmaceutical compositions comprising one or more compounds and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated, The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions,

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition, Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the present invention can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent.), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds of the present invention or an appropriate pharmaceutical composition thereof are effective, the compounds of the present invention can be administered in an effective amount. The dosages as suitable for this invention can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this invention can be to effect prophylaxis of recurring symptoms, For example, the dosage can be administered once or twice a day to prevent the onset of symptoms in patients at risk, especially for asymptomatic patients.

The absolute weight of a given compound included in a unit dose for administration to a subject can vary widely. For example, about 0.0001 to about 1 g, or about 0.001 to about 0.5 g, of at least one compound of this disclosure, or a plurality of compounds can be administered. Alternatively, the unit dosage can vary from about 0.001 g to about 2g, from about 0.005 g to about 0.5 g, from about 0.01 g to about 0.25 g, from about 0.02 g to about 0.2 g, from about 0.03 g to about 0.15 g, from about 0.04 g to about 0.12 g, or from about 0.05 g to about 0.1 g.

Daily doses of the compounds can vary as well. Such daily doses can range, for example, from about 0,01 g/day to about 10 g/day, from about 0.02 g/day to about 5 g/day, from about 0.03 g/day to about 4 g/day, from about 0.04 g/day to about 3 g/day, from about 0.05 g/day to about 2 g/day, and from about 0.05 g/day to about 1 g/day.

It will be appreciated that the amount of compound(s) for use in treatment will vary not only with the particular carrier selected but also with the route of administration, the nature of the condition being treated, and the age and condition of the patient. Ultimately the attendant health care provider may determine proper dosage.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, dose to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired, Administration can be enteral wherein the desired effect is systemic (non-local), composition is given via the digestive tract.

Administration can be parenteral, where the desired effect is systemic, composition is given by other routes than the digestive tract.

The compositions can include the compounds described herein in a “therapeutically effective amount.” Such a therapeutically effective amount is an amount sufficient to obtain the desired physiological effect, such as a reduction of at least one symptom of cancer.

This disclosure also includes methods for treating inflammatory diseases and autoimmune diseases comprising administering a therapeutically effective amount of at least one of the compounds described herein (e.g., compounds of formulae (I) and (II)) to a subject in need thereof. The types of autoimmune diseases that can be treated include, for example, rheumatoid arthritis, pancreatitis, mixed tissue connective disease, systemic lupus erythematosus, antiphospholipid syndrome, irritable bowel disease, type I diabetes mellitus, and Sjogren's disease.

As used herein, the terms “treat” and “treating” are not limited to the case where the subject (e.g. patient) is cured and the disease is eradicated. Rather, treatment that merely reduces symptoms, and/or delays disease progression is also contemplated.

The compounds and methods described herein can be used prophylactically or therapeutically. The term “prophylactic” or “therapeutic” treatment refers to administration of a drug to a host before or after onset of a disease or condition, If it is administered prior to clinical manifestation of the unwanted condition (e.g., disease or other unwanted state of the host animal) then the treatment is prophylactic, i.e.; it protects the host against developing the unwanted condition, whereas if administered after manifestation of the unwanted condition, the treatment is therapeutic (i.e., it is intended to diminish, ameliorate or maintain the existing unwanted condition or side effects therefrom). Administering the compounds described herein (including enantiomers and salts thereof) is contemplated in both a prophylactic treatment (e.g. to patients at risk for disease, such as elderly patients who, because of their advancing age, are at risk for arthritis; cancer, and the like) and therapeutic treatment (e.g. to patients with symptoms of disease or to patients diagnosed with disease).

The term “therapeutically effective amount” as used herein, refers to that amount of one or more compounds of the various examples of the present invention that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age; body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein,

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl; n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH2CH₂—, —CH₂CH2CH2CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example; alkyl, and also (C)alkyl, includes methyl and substituted methyl. As a particular example, (C)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro; thio, alkoxy, and halogen groups. As further example; representative substituted alkyl groups can be substituted one or more fluoro, chloro, bromo, iodo, amino, arnido, alkyl, alkoxy, alkylamido, alkenyl, alkynyl, alkoxycarbonyl, acyl, formyl, arylcarbonyl, aryloxycarbonyl, aryloxy, carboxy; haloalkyl, hydroxy, cyano, nitroso, nitro, azido, trifluoromethyl, trifluorornethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments; representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain, Thus, for example, “alkyl” also encompasses groups such as —[(CH₂)ᵣO]ₜH and the like, wherein each r is 1 2 or 3; and t is 1 to 500.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl, Examples of straight chain bi-valent (C₂-C₂₀)alkenyl groups include those with from 2 to 6 carbon atoms such as —CHCH—, —CHCHCH₂—, —CHCHCH₂CH₂—,and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl.

For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl, Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylarnido, alkenyl, alkynyl, alkoxycarbonyl, acyl, formyl, arylcarbonyl, aryloxycarbonyl, aryloxy, carboxy, haloalkyl, hydroxy, cyano. nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido, In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyi, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, Butyn-1-yl, butyn-2-yl, Butyn-3-yl, Butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatorns, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heteracyclyi group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

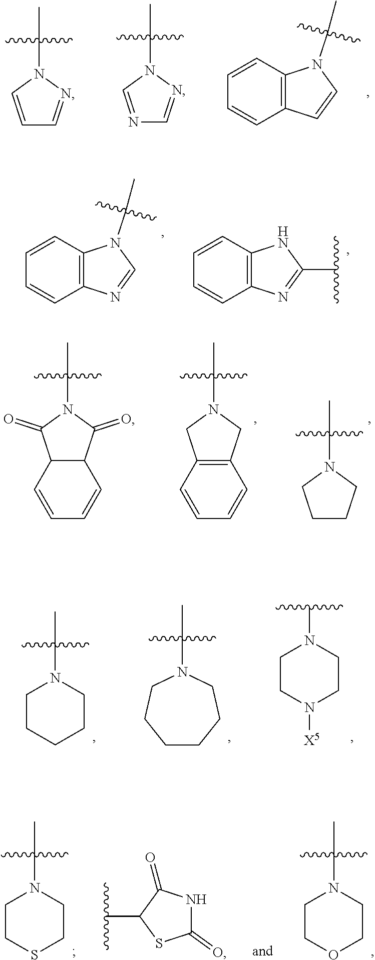

wherein X⁵ represents H, (C₁-C₂₀)alkyl, (C₆-C₂₀)aryl or an amine protecting group (e.g., a t-butyloxycarbonyl group) and wherein the heterocyclyl group can be substituted or unsubstituted, A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tent-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR₂, —NR₃⁺; wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylarnino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylarnido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “amido” as used herein refers to a group having the formula C(O)NRR, wherein R is defined herein and can each independently be, e.g., hydrogen, alkyl, aryl or each R, together with the nitrogen atom to which they are attached, form a heterocyclyl group.

The term “alkylthio” as used herein refers to a sulfur atom connected to an alkyl, alkenyl,or alkynyl group as defined herein,

The term “arylthio” as used herein refers to a sulfur atom connected to an aryl group as defined herein.

The term “alkylsulfonyl” as used herein refers to a sulfonyl group connected to an alkyl, alkenyl,or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amino group connected to two alkyl groups; as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, ROH (e.g., CH₂OH), OC(O)N(R)₂, ON, NO, NO₂, NO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R; N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl, (C₆-C₂₀)aryl, heterocyclyl or polyalkylene oxide groups, such as polyalkylene oxide groups of the formula —(CH₂CH₂O)_{f}—R—OR, —(CH₂CH₂CH₂O)_{g}—R—OR, —(CH₂CH₂O)_{f}(CH₂CH₂CH₂O)g—R—OR each of which can, in turn; be substituted or unsubstituted and wherein f and g are each independently an integer from 1 to 50 (e.g., 1 to 10, 1 to 5, 1 to 3 or 2 to 5).

Substituted also includes a group that is substituted with one or more groups including, but not limited to, the following groups: fluor( ) chloro, bromo, iodo, amino, amino, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro; azido, trifluoromethyl, trifluoromethoxy, thio, alkylthio, arylthiol, alkylsulfonyl; alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in; e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., compounds of the formulae (I) and (II)) can contain chiral centers, All diastereomers of the compounds described herein are contemplated herein, as well as racemates. Also contemplated herein are isotoporners, which are compounds where one or more atoms in the compound has been replaced with an isotope of that atom. Thus, for example, the disclosure relates to compounds wherein one or more hydrogen atoms is replaced with a deuterium or wherein a fluorine atom is replaced with an ¹⁹F atom.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, mak, tartaric, citric, ascorbic, pamoic, maleic, hydroxymaieic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed,, Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

Each embodiment described above is envisaged to be applicable in each combination with other embodiments described herein. For example, embodiments corresponding to formula (I) are equally envisaged as being applicable to formula (II),

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

One of ordinary skill in the art will recognize that the methods of the current disclosure can be achieved by administration of a composition described herein comprising at least one bronchodilator and at least one pulmonary surfactant via devices not described herein. Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure, Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### Examples

The disclosure can be better understood by reference to the following examples which are offered by way of illustration. The disclosure is not limited to the examples given herein.

### Methods and Materials

### Cell Lines and Reagents

The CellSensor® NF-κB-bla human monocytic THP-1 cell line was purchased from Thermo Fisher Scientific (Waltham, MA). THP-1 cells were purchased from American Type Culture Collection (ATCC, Manassas, VA). Cells were cultured in RPMI medium (Thermo Fisher Scientific, Waltham, MA) supplemented with 10% FBS (Omega Scientific Inc., Tarzana, CA), 100 U/mL penicillin, 100 μg/mL streptomycin (Thermo Fisher Scientific, Waltham, MA), and 55 μM ßmercaptoethanal (SigmaAldrich, St. Louis, MO).

Rheumatoid arthritis fibroblast-like synoviocytes (RA FLS) were isolated from synovial tissues derived from patients with RA when they underwent joint replacement surgery or synovectomy. Patients were age ≥18 years with active RA based on the ACR 1987 Revised Criteria and consent forms were completed by the patients before surgery. The study protocol was approved by the institutional review board at TokyoMedical andDental University, Tokyo, Japan and are in accordance with the principles of the Declaration of Helsinki. RA FLS were cultured in DMEM supplemented with 10% FBS, 100 U/mL penicillin, and 100 μg/mL streptomycin in a humidified 5% CO2 incubator. All experiments used proliferating RA FLS.

LPS (Escherichia coli 0111:B4, Sigma-Aldrich, St Louis. MO) was used in the HTS, and LPS-EB Ultrapure (InvivoGen, San Diego, CA) was used in the confirmation screens and subsequent studies. Human TNF (Thermo Fisher Scientific, Waltham, MA and R&D systems, Minneapolis, MN), IL-1ß (Promega, Southampton, United Kingdom), dexamethasone (DEX, Fresenius Kabi Usa, Lake Zurich, IL and MP Biomedicals, Solon, OH) and 5-(4-fluorophenyl)-2-ureidothiophene-3 carboxylic acid amide (UTC; Toronto Research Chemicals, Inc., Ontario, Canada), a known IKK inhibitor (Endo et al., 2007) were commercially purchased.

Hit compounds were purchased from ChemBridge (San Diego, CA) and ChemDiv (San Diego, CA) and dissolved in dimethyl sulfoxide (DMSO, Sigma Aldrich, St Louis. MO; Supplemental Table S1). Purity of the compounds was verified as >95% by LC-MS. Endotoxin levels were less than 10 EUIpmol by EndoSafe® (Charles River Laboratory, WImington, MA).

### NF KB Activation Assay Using Reporter Cells

The CellSensor® NF-κB-bla THP-1 cell line has a stably integrated ß lactamase reporter gene under the control of the nuclear factor kappa B (NF-κB) response element. LPS induced NF-κB activation resulted in ß-lactamase production. In the absence of ß-lactamase activity, excitation of the coumarin at 409 nrn in the ß-lactamase substrate (LiveBLAzerTM-FRET BIG (CCF4-AM), Thermo Fisher Scientific) resulted in emission at 520 nm. In the presence of lactamase, CCF4 was enzymatically cleaved and excitation at 409 nm produced a blue fluorescence signal (at 450 nm), The CellSensor® NF-κB-bla THP-1 cells were dispersed in 96-well plates (5×104 cells/200 μL/well) and incubated for 4 h, Then the cells were treated with 5 μM of each compound and 10 ng/ml of LPS for 5 or 12 h in 5% CO2 at 37° C. After incubation, the ß-lactamase substrate mixture (prepared according to the manufacturer's protocol) was added to each well. Plates were incubated at room temperature in the dark for 2 h. Fluorescence was measured on a Tecan Infinite M200 plate reader (Tecan, Mannedorf, Switzerland) at an excitation wavelength of 405 nm and emission wavelengths of 465 and 535 nm. Emission ratios are calculated by dividing values from emission wavelength of 465 nm by those from emission wavelength of 535 nm, The response ratio was calculated as follows [(emission ratio of a test well)/average emission ratio of wells with vehicle (0.5% DMSO)] and values were normalized to the LPS control treated wells [response ratio of the compound/response ratio of LPS].

### Cell Viability Assays

Two types of tetrazolium were used for viability assays. For THP-1 cells, the cells were dispensed in 96-well plates (105 cells/200 μL/ well) and treated with 5 Oil of each compound. After 18 h of incubation, 0.5 mg/ml 3[4,5-dimethylthiazol-2-yl]-2,5-dipheyl tetrazolium bromide (MTT; Thermo Fisher Scientific) in assay media was added to each well at a final concentration of (0.5 mg/ml) and further incubated for 4-6 h. The absorbances at 570 and 650 nm were measured by a Tecan Infinite M200 plate reader, For RA FLS, the cells were dispersed into 96-well flat bottom plates (104/200 μL/well) and incubated overnight. The next day, RA FLS were treated with 5 μM of each compound for 18 h of incubation. Ten μl of WST-8 [2-(2-methoxy-4-nitrophenyl)-3-(4-nitrophenyl)-5-(2,4-disulfophewnyl)-2H-tetrazolium, monosodium salt] solution was added to each well and the cells were further incubated for 2-4 h. The absorbance was measured at 450 nm with Bio-Rad iMark plate reader (Bio-Rad Laboratories Inc., Hercules, CA, Unites States).

For apoptosis studies, THP-1 cells (105 cells/200 μL/well) were plated in 96 well plates and pre-treated with 5 pM compound, 5 pM DEX, or 5 pM UTC; or vehicle (DMSO) for 1 h and then LPS (10 ng/ml) was added. After 24 h incubation the cells were washed twice with cold PBS and then resuspended in Annexin V Binding Buffer (BD Pharmingen, Mountain View, CA) and 5×104 cells in 50 pL were transferred to a V bottom plate. The cells were stained with FITC-Annexin V (BD Pharmingen) and 7-Amino-Actinomycin (7-AAD; BD Pharmingen) for 15 min and evaluated by flow cytometry (MACSQuant® Analyzer 10, Miltenyi Biotec, Germany). Data were analyzed using FlowJo software (FlowJo LLC, Ashland, OR).

### Cytokine and Chemokine Production Assays

THP-1 cells (105 cells/200 μL/well) and RA FLS (104/200 μL/well) were plated in 96 well plates and pre-treated with compound or vehicle for 1 h and then stimulated with either LPS (10 ng/ml), 1L-1ß (2 ng/ml) or TNF (2 ng/ml for THP-1 and 1 ng/ml for RA FLS). After 18 h of incubation, supernatants were collected and the levels of cytokines and chemokines in the culture supernatants were measured by ELISA according to the manufacturer's protocols (R&D systems, Minneapolis, MN).

### Compound Clustering into Chemotypes

The structures of the compounds (simplified molecular-input line-entry system format, SMILES) were subjected to substructure-based clustering using the server based ChemMine tools (University of California; Irvine; http://chemmine.ucr.edu/tools/launch job/Clustering/) and binning clustering application with a similarity cutoff of 0.5.

### Drug Synergy Analysis

Drug synergy analysis was performed using lsobologram plots which were calculated according to reported procedures to compute IC50 and determine synergism. IC₅₀ is computed from the median effect equation. Synergism analysis is carried out using the Combination index (Cl)-isobol method. Data analysis was performed using the CompuSyn software available on combisyn.com, Detailed methodology was used as described in a prior report. Briefly, the median-effect equation is computed to obtain the linear regression for the effect of inhibitors DEX and 1−1 as Fₐ/Fᵤ=(D/D₅₀)\hat{ }m, where D is the dose; Fₐ and Fᵤ is the fraction of the inhibition and uninhibited response by the dose D (Fₐ+Fᵤ=1); D₅₀ is the dose producing the median effect (i.e, IC₅₀). The constant m determines the shape of the dose-effect curve. The median-effect equation in logarithmic form is log (Fₐ/Fᵤ) =m log(D)−m log (D₅₀) which essentially represents a linear relationship between log(Fₐ/Fᵤ) and log(D). Thus, linear regression curves are obtained with the observed inhibition data for the individual inhibitors to obtain estimated values for the parametersmand D₅₀. This is followed by Cl-isobol method to quantitatively assess the synergism between these inhibitor drugs. A combination index (Cl) is estimated from dose-effect data of single and combined drug treatments. A value of C; less than one indicates synergism; Cl=1 indicates additive effect; and Cl>1 indicates antagonism. Drug interaction (synergism or antagonism) is more pronounced the further a CI value is from 1. Formally, the combination index (Cl) of a combined drug treatment is defined as Cl=D₁/Dx₁+D₂/Dx₂Here D₁and D₂ are the doses of DEX and 1−1, respectively, in the combination; Dxi and Dx2 each is the dose of a treatment with only DEX and 1−1 that would give the same effect as that of the combination, respectively. The doses Dx₁ and Dx₂ were estimated from the median effect equation above for single drug treatments. From the median effect equation, the estimated dose (i.e., D) necessary to produce the inhibition (i.e., Fₐ, Fᵤ) obtained by the combination was calculated. The results are presented as a normalized isobolograrn. A point in the isobologram represents the effect of a drug(s) treatment. The further a point lies from the additive line, the larger the difference between one and its Cl, hence the stronger is the synergistic effect.

### Example 1: Overall Screening Strategy and Design

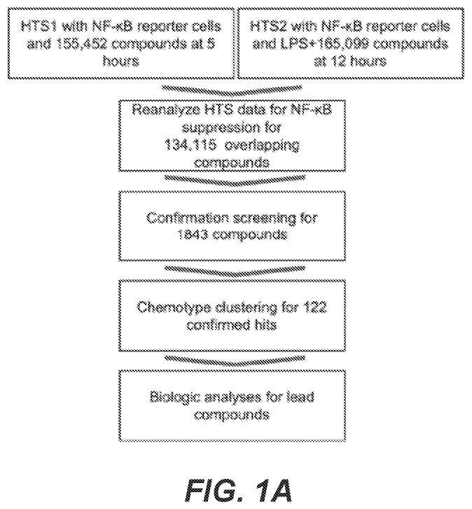

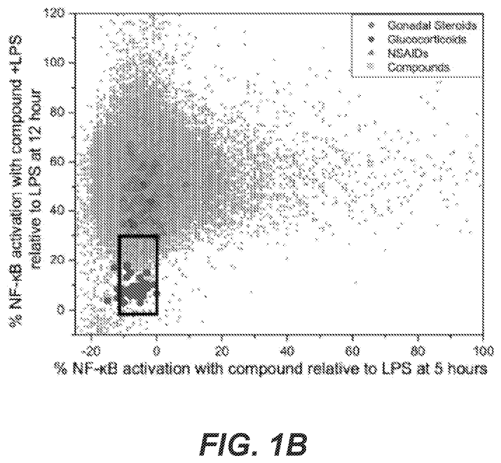

As part of a compound identification strategy (FIG. 1A) data from two prior HTS were re-analyzed that had previously conducted using CellSensor NF-κB-bla THP-1 reporter cells and compound libraries that were acquired at two different times (5 years apart) from the University of California, San Francisco, Small Molecule Discovery Center (SMDC: https://smdc.ucstedu). An area was determined that bounded the activities of the named GCs in overlapping subset of the libraries and identified 1824 compounds that attenuated NF-κB activities in both HTS within the perimeter of this area (box in FIG. 1B). A series of confirmation screens were performed with these hit compounds (5 μM) for their effects on the kinetics of NF-κB activity in LPS stimulated reporter cells at peak (5 h) and decay (16 h) timepoints. There were 122 compounds that met the following criteria: NF-κB activity <50% max at 5 h or <25% max at 16 h. These compounds were then evaluated for effect on 1L-8 production and cellular toxicity by MTT assay in THP-1 cells. Excluding compounds with <90% viability by MTT assay, the remaining compounds were clustered into chemotype families. Candidate compounds were purchased from commercial vendors to represent chemotype families with the largest number of active members and to represent chemical diversity, After purchasing candidate compounds, further biological activities using THP-1 cells and synovial fibroblasts were analyzed from rheumatoid arthritis patients (RA FLS) for primary activity and potential synergism with dexamethasone,

### Example 2: Re-analysis of Existing High Throughput Screening Data

In prior studies, two HTS were conducted to identify novel compounds that initiated or sustained innate immune activation via the NF-κB pathway using CellSensor NF-κB-bla reporter containing THP-1 cells. The libraries for these studies came from the SM DC and had 134,115 overlapping compounds and LPS was used as a control on each plate in both studies. The reporter cells were incubated with compound alone (5 pM) for 5 h in the first HTS (HTS1). In the second HTS (HTS2), the reporter cells were incubated with compound (5 μM) in the presence of LPS (100 ng/ml) for 12 h as a primary stimulus. The FRET activity was normalized to the LPS controls on each plate in the respective HTS. The normalized activities of individual compounds were plotted for the activities after 5 h (compound alone in HTS1, x-axis) or 12 h (compound+LPS in HTS₂, y-axis) incubation (FIG. 1B). The chemical collection from the SMDC contained compounds with known drug properties including several GCs, gonadal steroids and cyclo-oxygenase (COX) inhibitors. To segregate compounds with the most potential as immunosuppressants, the compounds with similar activity to the glucocorticoid cluster region were chosen and were excluded from the region populated with non-steroidal anti-inflammatory drugs (NSAIDS) or gonadal steroids for further screening (FIG. 1B), A total of 1824 compounds were selected from the glucocorticoid region of the combined HTS data that fit the activation thresholds set at each respective time point.

### Example 3 Confirmation Screen and Kinetic Profiling of NF-κB Activity

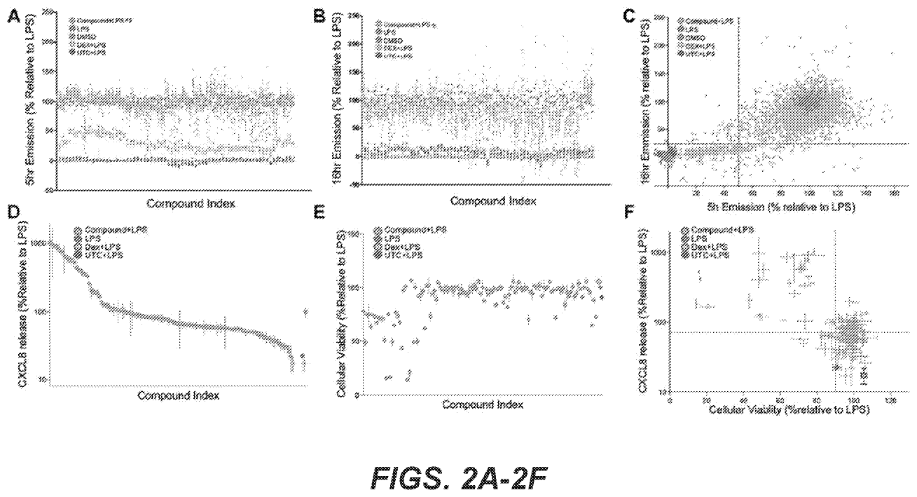

The 1824 compounds that were identified by the data mining strategy to reduce NF-κB signaling were then rescreened in duplicate using the same THP-1 CellSensor NF-κB-bla reporter cells. In prior work conditions were established where LPS (10 ng/ml)-induced NF-κB activity which peaked at 5 h and decayed to 60% at 16 h after stimulation. Using LPS as a primary inflammatory stimulus the effect of these compounds on NF-κB activity was evaluated at two time points to profile the kinetics of their suppression. Analysis of the compound behavior at the 5 h time point against the 16 h time point shows a higher number of compounds with lower NF-κB activity following a longer incubation time vs. the shorter time (FIG. 2A,B). These confirmation screens included dexamethasone (DEX), and 5-(4-fluorophenyl)-2-ureido-thiophene-3 carboxylic acid amide (UTC) as controls that suppress NF-κB activity with distinct mechanisms. As expected, DEX inhibited NF-κB activity more potently at 16 h than 5 h, and UTC inhibited NF-κB activity at both time points.

To select possible immunosuppressants as hit compounds a naive standard activity-based approach was utilized by selecting compounds with a defined activity threshold (frequently called a “Top X” approach). The known bioactive compounds were excluded and then set the desired activity threshold levels of NF-κB activity at 5 50% or 5.25% of the normalized FRET emission ratios at 5 and 16 h, respectively (FIG. 2C). This area encapsulated most of the DEX controls and 122 unique compounds which were selected for further assessment (FIGS. 2D-F).

### Example 4: Cytotoxicity and Suppression of Chemokine Production in THP-1 Cells

The 122 hit compounds underwent additional screening and were evaluated for their effects on cell viability and the ability to suppress production of an NF-κB-associated chemokine, CXCL8 (IL-8) by LPS stimulated THP-1 cells (FIG. 2D). The cells were also examined for viability after 24 h of stimulation by MTT assay (FIG. 2E). Relative CXCL8 production and cellular viability by the treatment candidate compounds, DEX, and UTC were normalized to the LPS vehicle controls (FIGS. 2D-F), Compounds that showed low cytotoxicity (>90% viability) and suppressed CXCL8 production at 70% or lower relative to the control were brought forward as potential candidates for future evaluation as immunosuppressants (FIG. 2F). The 122 hit compounds were also tested for apoptosis induction in the presence of LPS, The % live cells in the apoptosis assay and the % viable cells in the MTT assay correlated with a Pearson r coefficient of 0.85 (p<0.0001), However there were six compounds that demonstrated >90% viability in the MTT assay, but had >10% apoptotic cells in the apoptosis assay indicating that the two assays provided complementary assessments.

### Example 5: Chemotype Clustering and Validation of Lead Compounds

Of the 122 hit compounds 74 were clustered into 18 chemotypes based on their molecular similarities and common scaffolds using Tanimoto indexes and 48 compounds were singletons (compounds not associated with a chemotype). The 51 compounds that met all of the biological selection criteria (90% viability and 70% CXCL8 release) consisted of 11 chemotypes and included 17 singletons (Table 1).

TABLE 1
Number of compounds in lead chemotype clusters by screening stage.

| Chemotype cluster | | Number in 1824 | Number passed all | Selection or exclusion |
|---|---|---|---|---|
| number | Chemotype | starting compounds | selection criteria | criteria |
| 1 | 1H-pyrazolo [3,4 d] pyrimidin-4-amine | 22 | 11 | Selected |
| 2 | (E)-5-benzylidene imidazolidine-2,4-dione | 17 | 4 | Excluded as a michael acceptors |
| 3 | Bis-aryl urea | 12 | 1 | Selected |
| 5 | 2-Nitro furan arylamide | 9 | 5 | Selected |
| 6 | Piperidine derivatives | 9 | 2 | Weak inhibition |
| 8 | (E)-3-phenyl-1-(quinolin-3-yl)prop-2-en-1-one | 8 | 3 | Excluded as a michael acceptor |
| 10 | 1H-pyrazolo [3,4-b] quinolin-3-amine | 6 | 2 | None commercially available |
| 13 | 2-Imino-1,2-dihydro-5H-dipyrido | 5 | 3 | Selected |
| | [1,2-a [symbol] 2′ [symbol] 3′-o]pyrimidin-5-one | | | |
| 15 | Piperazine derivatives | 5 | 1 | None commercially available |
| 16 | (E)-3-phenyl-1-(pyridine-3-yl)prop-2-en-1-one | 3 | 1 | Excluded as a michael acceptors |
| 17 | Thiophene pyrazine piperidine analogs | 3 | 2 | Selected |
| 18 | Pyrazole derivatives | 22 | 2 | None commercially available |
| n/a | Singleton compounds | n/a | 14 | Excluded |

[symbol]

indicates data missing or illegible when filed

TABLE 2
Validation of purchased candidate compounds.

| | | | CXCL8 production. | CXCL8 production. | |
|---|---|---|---|---|---|
| | | | Relative to | Relative to | % Cellular |
| Compound | | MW | LPS^{b} | LPS^{c} | Viability |
| number | Chemotype | (g/mol) | (original library) | (purchased) | (Purchased) |
| 1-1^{a} | 1H-pyrazolo [3,4 d] pyrimidin-4-amine | 321 | 50. [symbol] % | 47.1% | 98.7 |
| 1-2^{a} | 1H-pyrazolo [3,4 d] pyrimidin-4-amine | 295 | [symbol] 2.7% | 70.1% | 100.1 |
| 3-1^{a} | Bis-aryl urea | 261 | 50.6% | 60.7% | 105.6 |
| 5-1^{a} | 2-Nitro furan arylamide | 276 | 21.9% | 18.8% | 98.7 |
| 5-2^{a} | 2-Nitro furan arylamide | 300 | 54.3% | 29.8% | 101.8 |
| 5-3^{a} | 2-Nitro furan arylamide | 312 | 66.7% | 37.7% | 100.1 |
| 13-1 | 2-Imino-1,2-dihydro-5H-dipyrido [1,2-a [symbol] 2′ [symbol] 3′- | 4 [symbol] 4 | 25.8% | 96% | 106.1 |
| | d]dipyrimidin-5-one | | | | |
| 13-2 | 2-Imino-1,2-dihydro-5H-dipyrido [1,2-a [symbol] 2′ [symbol] 3′- | 4 [symbol] 4 | 32.3% | 124.8% | 104 |
| | d]dipyrimidin-5-one | | | | |
| 13-3 | 2-Imino-1,2-dihydro-5H-dipyrido [1,2-a [symbol] 2′ [symbol] 3′- | 4 [symbol] 4 | 54.8% | 129% | 101.4 |
| | d]dipyrimidin-5-one | | | | |
| 17-1 | Thiophene pyrazine peperidine analog | 417 | [symbol] 0.1% | 82.2% | 91.3 |

^{a}Candidate compounds validated in rescreening.
^{b}The mean CXCL8 induced by LPS was 185.3 pg/ml and was normalized as 100%.
^{c}The mean CXCL8 induced by LPS was 201.1 pg/ml and was normalized as 100%. [symbol]
indicates data missing or illegible when filed

Three of the eleven chemotypes were excluded based on the potential for them to be Michael acceptors, In many cases α,β-unsaturated carbonyls such as those found in these chemotypes can form adducts with thiols, especially under physiological conditions (pH=6-8), reducing the in vivo efficacy. Also, Michael acceptors are often reversible IKKß inhibitors and thereby inhibit NF-KB response. Thus, from the remaining chemical families, ten compounds from 5 chemotypes were purchased to validate and further assess as they had multiple hits within the chemotype cluster and represented chemical diversity between the scaffolds. These compounds were reassessed for their suppression of CXCL8 production and cytotoxicity in THP-1 cells (Table 2). Six inhibitors from three chemotypes reduced the level of CXCL8 levels to 70% or less than that of the LPS control. However, the other four compounds from chemotypes 13 and 17 did not meet the set criteria and were inconsistent with the data previously seen from the compounds obtained from the original HTS library. These discrepancies could be due to the age of the DMSO stocks in the HTS samples permitting degradation, precipitation or other unknown modifications. LC-MS analysis of the purchased compounds showed that the purity of the material (>95% by HPLC) was sufficient to validate the observed activity.

### Example 6: Dose-Response of Lead Compounds on Cyto/Chemokine Production in Stimulated THP-1 Cells

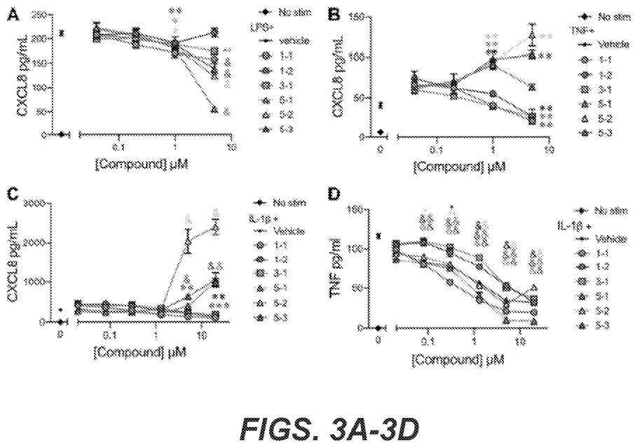

The lead compounds belonging to chemotypes 1, 3, and 5 were tested for potency in suppressing chemokine and cytokine production by THP-1 cells in the presence of different inflammatory stimuli. The production of CXCL8 induced by either LPS (10 ng/ml), IL-1ß (2 ng/ml), or TNF (2 ng/ml) and TNF induced by IL-1ß (2 ng/ml) was assessed using serially diluted compounds. The compounds except 1-2 reduced the level of CXCL8 production stimulated by LPS in a dose dependent manner (FIG. 3A). However, the compounds from chemotype 5 enhanced CXCL8 release by THP-1 cells when stimulated with IL-111, or TNF (FIGS. 3B, C). The IC₅₀ for 1-1, 1-2 and 3-1 for TNF stimulated CXCL8 release included 900 nM, 4,130 and 960 nM respectively (FIG. 38). The 1Cs0 for 1-1, 1-2 and 3-1 for IL-1ß stimulated CXCL8 release included 400, 1770, and 2020 nM, respectively, (FIG. 3C). Interestingly, all of the compounds including those from chemotype 5 reduced TNF release by THP-1 cells when stimulated with IL-1F3 (FIG. 3D). The IC₅₀ for 1-1, 1-₂, and 3-1 for IL-1ß stimulated TNF release included 190, 2,770, and 2,420 nM, As an inflammatory tissue environment can have a variety of perpetuating stimuli it was opted not to move forward with the compounds from chemotype 5 as they may increase inflammation under certain circumstances.

### Example 7: The Interaction of Lead Compounds and Dexamethasone

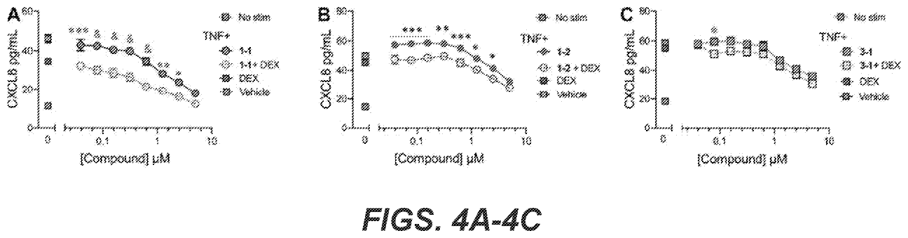

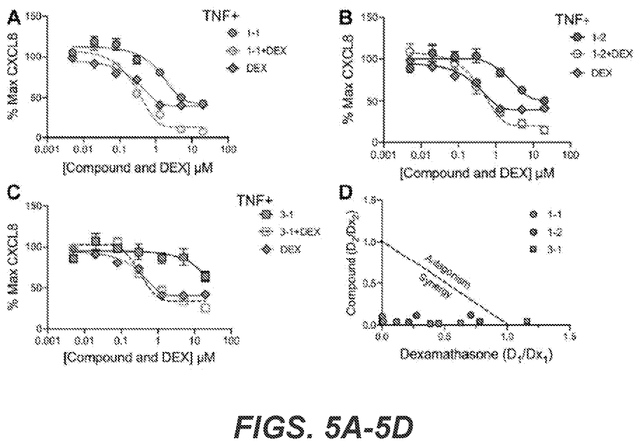

To assess whether the remaining candidate compounds would provide additional benefit to a low dose of glucocorticoid, THP-1 cells were stimulated with TNF and treated them with serially diluted compounds and 100 nM DEX (FIGS. 4A-C). The addition of DEX significantly reduced the CXCL8 production at all of the effective doses of 1-1, but was only effective at the lower doses of 1-2. There was minimal benefit to the effect of 3-1. Next whether there was an additive or synergistic effect with these three compounds and DEX was formally addressed. The compounds and DEX were titrated at the same molarity and in culture with TNF stimulated THP-1 cells and the release of CXCL8 was measured (FIGS. 5A-C), Here the 1050 for 1-1 and dexamethasone for TNF stimulated CXCL8 release were 968 and 300 nM respectively (FIG. 5A). The four CXCL8 levels below the maximum plateau were used to calculate an isobologram (FIG. 5D). The relative potency values for compound 1-1 are near the origin, demonstrating synergy. The values for 1-2 were modestly synergistic, however the values for 3-1 were not all consistent with synergy. Hence compound 1-1 was considered for testing in primary human cells (FIG. 5D).

### Example 8: Compound 1-1 Suppresses Chemokine Production by RA FLS and Is Synergistic With GC

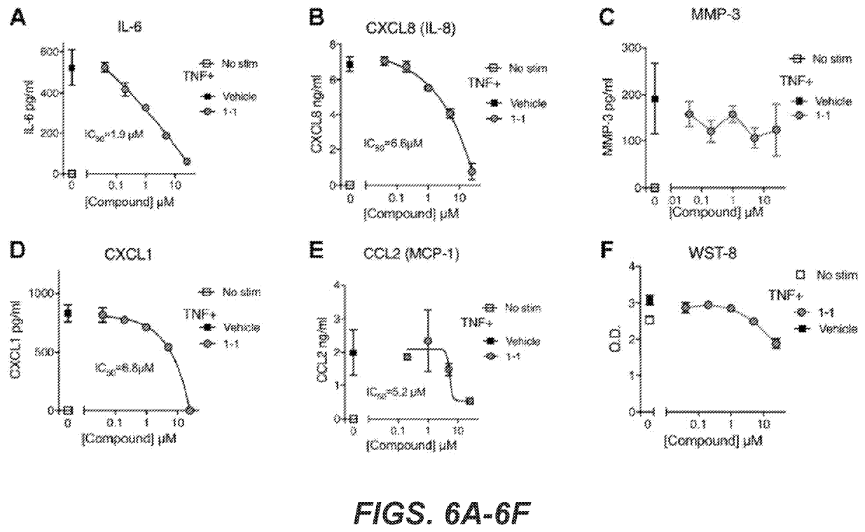

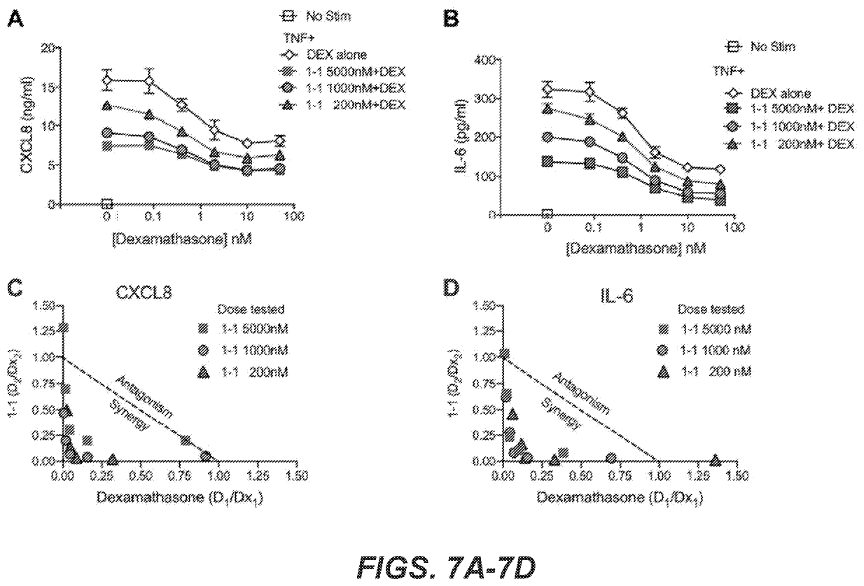

In the pathogenesis of rheumatoid arthritis, fibroblast-like synoviocytes (RA FLS) are a primary source of inflammatory cytokines and chemokines in inflamed joints. The immunomodulatory effects and cytotoxicity of compound 1-1 on RA FLS was analyzed. Compound 1-1 dose-dependently suppressed CXCL1 CXCL8, CCL₂, and IL-6 production induced by TNF, but not MMP-3 production (FIGS. 6A-E). Compound 1-1 also showed low cytotoxicity in RA FLS, similar to the THP-1 cells (FIG, 6F). To assess the synergistic effect of compound 1-1 with DEX in RA FLS, IL-6 and CXCL8 suppression was analyzed by co-titrating DEX and compound 1-1 in cultures with TNF stimulated RA FLS (FIG. 7A, B). Isobolograms of the potency ratios indicated that compound 1-1 also showed synergistic effects with DEX in RA FLS for both IL-6 and CXCL8 release (FIGS. 7C, D).

### Discussion

Recently there has been significant development of biologic and non-biologic disease modifying anti-rheumatic drugs (DMARDs), which have moved into clinical application. However, GCs and NSAIDs remain indispensable as bridge therapy or co-therapy with DMARDs. Here, compounds that reduced NF-κB activity and chemokine/cytokine secretion induced by potent inflammatory stimuli, and acted synergistically with GCs, were identified. Compounds were selected based on a classic “Top X” approach for bioactivity, but the selection of lead candidates was also informed by the frequency of hits in the larger chemotype clusters. Hit confirmation rates were previously improved by using a similar chemoinformatic enrichment method for hit selection. These compounds were not toxic to the monocytic cell line or to primary human cells in culture.

In the past a broad cell based screening approach was used to identify compounds with a desired function and did not limit the potential targets. By re-analyzing the data from two existing HTS assays, 1824 candidate compounds with activity similar to GCs from the >134,000 compounds that overlapped between the two HTS libraries were identified. Once these 1824 compounds were selected, their effects on the kinetics of NF-κB activity at two different time points, which had been identified as peak activity for the dose of LPS chosen and then later in the decay phase of the LPS stimulation, were reassessed. There were known NF-κB inhibitory compounds included in the library, including IKK inhibitors and polymyxin B, which suppressed NF-κB activity at both timepoints. However, the GC in the library suppressed NF-κB activity only at the later time point and not at the peak LPS stimulated NF-κB activity. Hence 122 compounds were chosen using a Top X selection approach that suppressed NF-κB activity at one or both time points.

Cell-based phenotypic assays generally rely on multiple biological pathways to show the desired effect and can be prone to false positives. However, confidence in compound selection was increased by adding a chemoinformatic approach and clustered the compounds by scaffold (chemotype). The advantages of this approach had been demonstrated in previous reports, that is, a large cluster suggests that there are replications in favorable biological activity of the candidate compounds, and the negative data afford a structure-activity relationship within the family to guide future strategic structure-activity designs. Interestingly, chemotype cluster #1 was the largest one in the chemical library. Although the molecular target has not yet defined, which is a limitation of using a cell-based assay, some good guidance is provided by 11 hit compounds and more than a hundred compounds with a shared chemotype in the larger library that lacked suppressive activity for future structure-activity experimental design.

The lead compound 1-1 has been previously described to have bioactivity in another system. This compound was discovered as one of a chemotype cluster of pyrazolo [3,4 d]pyrimidines to be a positive allosteric modulator of the metabotropic glutamate receptor subtype 4 (mGluR4). Metabotropic glutamate (mGlu) receptors are a family of G protein-coupled receptors activated by the neurotransmitter glutamate. This activity, including modulation of Ca²⁺ flux, was characterized in cellular experiments. Although direct binding to a target was not performed, others have indicated that a different mGLU4 positive allosteric modulator (PAM) could inhibit TNF release from LPS stimulated microglial cells in culture. Other cell types such as dendritic cells may be affected by this class of drug. A PAM of mGlu4 has been demonstrated to activate noncanonical mGluR⁴ signaling in dendritic cells (DC) and induce a tolerogenic functional phenotype through IDO1, an immunoregulator and reduce neuroinflammation in a murine model of multiple sclerosis.

As many small molecules have multiple targets with different binding affinities and this may be the case with 1-1. The premise that a compound that reduced NF-KB signaling may be beneficial to lower the dose of steroids needed to attain an anti-inflammatory effect was at the start of the methods described herein. GCs bind the glucocorticoid receptor (GR) and form a GC-GR complex when they transition into the nucleus, and then regulate gene expression by transactivation (TA) with binding of GC-GR complex to gene promoters, and by transrepression (TR). Since most of the adverse effects induced by GCs were mediated by metabolic effects via TA by GC-GR complex, several groups tried to identify selective GR activators (SEGRA) from natural products by assessing their binding to GR and their activity in transactivation and transrepression assays. As described herein, lead compounds showed similar inhibitory kinetics with GCs, suggesting that an inhibitory mechanism might be shared with GCs. However, the lead compound did not inhibit all NF-κB associated activity as seen in the minimal inhibition of MMP-3 production by FLS. The promotor region forMMP-3 includes binding sites for the activator proteins (AP)-1, the polyomavirus enhancer-A binding protein-3 (PEAS), and other transcription factors that may continue to induce MMP-3 transcription despite partial NF-κB inhibition. Identifying the mechanism of action of the compounds described herein and comparing with GCs would be the next step to further drug development.

To minimize the adverse events of GCs, decreasing the dosage of GCs with the concomitant use of other agents was examined to maintain the therapeutic efficacy. The lead compound 1-1 clearly demonstrated synergistic effects with DEX (FIGS. 5-7), suggesting that this compound may have a dose-sparing effect. These findings indicate the possibility of reducing the dose of GCs, but also potentially enhancing the effects of endogenous GCs secreted physiologically. Other agents have been reported to reduce inflammation in models of arthritis that are insufficient alone, but utilize a complementary pathway that favorably modulates the activity of a known therapeutic agent. For example, the receptor tyrosine phosphatase sigma (PTPRS) activating decoy protein attenuated severity of arthritis when combined with low dose of a TNF inhibitor, but was insufficient in itself to have an effect. In addition, the combination of an inhibitor of cell proliferation and a TNF inhibitor exerted synergistic effects without reducing immune responses.

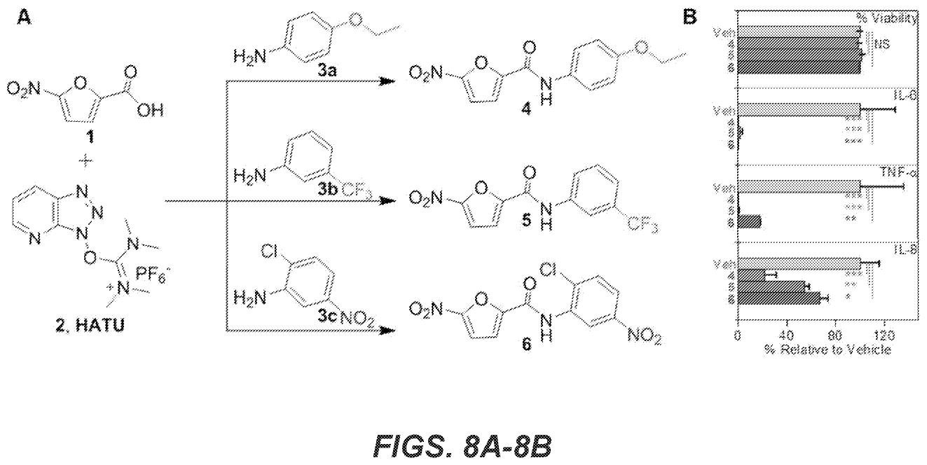

In parallel, nitrofuranylamide compounds were explored belonging to group 5 for potency to reduce allodynia and arthritic pain. Selected three nitrofuranylamides (4, 5, and 6) based on their potency to suppress inflammatory cytokines such as IL-8 in the presence of LPS in THP-1 cells and toxicity measured as reduced cell viability using an MTT assay were synthesized (FIG. 8A) using a one-step amide bond forming coupling reaction using HATU (Hexafluorophosphate azabenzotriazole tetramethyl uronium, 2) reagent using three different anilines (3a-3c) and a common reagent 2-nitrofuran-5-carboxylic acid (1). To confirm the biological activities of the synthesized compounds, the compounds were assayed for suppression of LPS induced cytokine (IL-S, TNF-α and IL-8) production in THP-1 cells compared to vehicle (Veh) confirming their anti-inflammatory potencies. The cellular viability (measured as % Viability compared to Veh) was approximately 100% suggesting these compounds were relatively non-toxic (FIG. 8B).

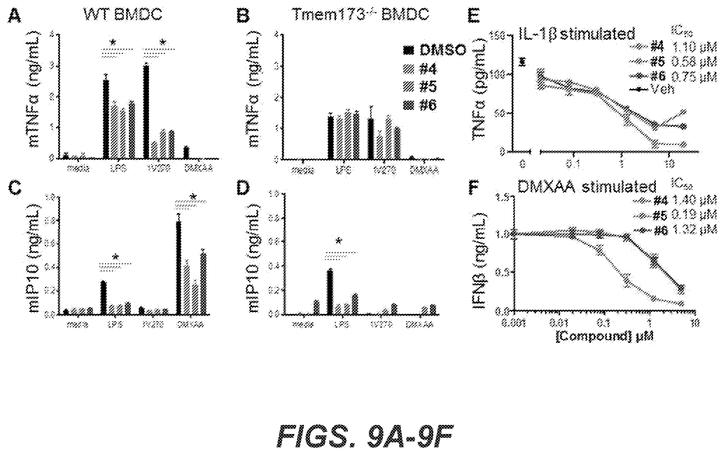

Since the synthesized compounds were identified via assays in human cells, there was interest in verifying the potency of these compounds in murine cells. Compounds were incubated with murine bone marrow-derived dendritic cells (BM DCs) obtained from wild-type (WT) mice and STING null (Tmem173^{-/-}) mice as STING had been reported as the target of other nitrofuranylamides. All the compounds inhibited the induction of murine TNF-α and IP-10 induced by different TLR stimuli including TLR-4 agonist LPS, TLR-7 agonist 1V270 and murine-specific STING agonist DMXAA. The Tmem173^{-/-} cells also showed loss of activity by these compounds except for LPS induced IP-10 induction, suggesting partial involvement of STING and TLR-4 signaling pathways for the activity of these compounds (FIGS. 9A-9D). To compare these compounds, the IC₅₀ values of these compounds were assessed in dose titration studies. In both human and mouse cell studies compound 5 had cytokine inhibition IC₅₀ values in the nanomolar (nM) range, Shown are examples of cytokine TNF-a inhibition studies performed in THP-1 cells stimulated with IL-1β (FIG. 9E) and IFNββinhibition studies performed in mBMDCs stimulated with DMXAA (FIG. 9F).

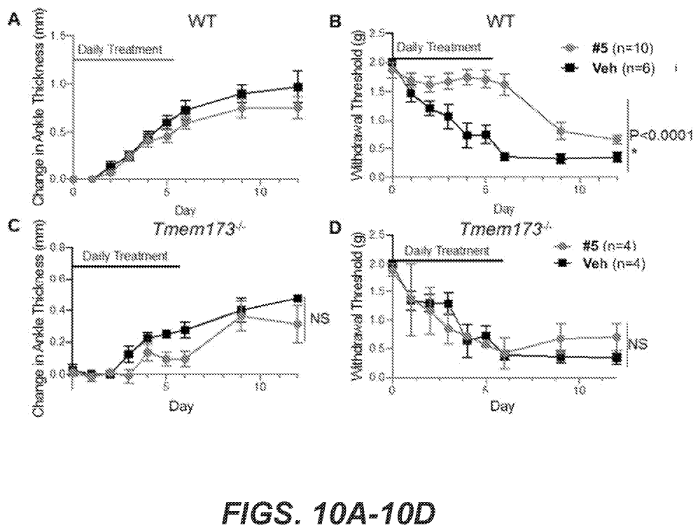

Encouraged by these results and based on a recent report that such bioactivity possessing compounds could be useful for the treatment of nociceptive pain and allodynia, compound 5, which was chosen based on the higher potency to suppress inflammatory cytokines compared to compounds 4 and 6, was evaluated for proof-of-concept in a mouse model of arthritis. Compound 5 or vehicle (Veh) was injected into mice (n=10 for compound 5 and n=6 for Veh) intraperitoneally (IP) at 750 nmoles concentration twice a day (BID) for 6 consecutive days, Inflammation was assessed as ankle thickness, while mechanical allodynia was tested by von Frey filaments, Compound 5 had modest effect on acute inflammation in the ankle, but significantly reduced mechanical allodynia (FIGS. 10A-B). The compound had no statistically significant effect on pain or swelling in Tmem173^{-/-} mice, but greater numbers would be needed as there was a slight trend (FIGS. 10C-D). In vivo testing with a representative nitrofuranylamide compound 5 indicated that the potent compound in the scaffold abrogates the onset of allodynia and can partially reverse established mechanical allodynia during the time period that the drug is administered with the modest effects on paw swelling.

In summary, the methods described herein identified novel anti-inflammatory compounds and compounds for allodynia treatment by an immune phenotype based screening. The lead compounds showed anti-inflammatory effects with minimal if any cellular cytotoxicity. By analyzing multiple potential inflammatory stimuli, including LPS. TNF and L-1ß the candidates were narrowed to those that reduced chemokine secretion to all tested stimuli. The lead 1H-pyrazolo [3,4 d] pyrimidin-4-amine compound (1-1) had an IC₅₀ at the micromolar level in RA FLS comparable to that in the human monocytic cell line THP-1. Furthermore synergistic anti-inflammatory effects with dexamethasone and compound 1-1 were demonstrated in both THP-1 cells and primary human RA FLS. The studies described herein provide the foundation for future studies including specific mechanism of action studies, target identification, and additional preclinical assessments of FLS migration, invasion, proliferation and apoptosis should be performed using the lead compound. A synoviocyte-directed therapy as evaluated here with compound 1-1 combined with a targeted biologic strategy, like an anti-TNF monoclonal antibody, could be a successful strategy with less toxicity than current therapeutic approaches, Additionally, some nitrofuranylamide compounds were found to be potent in reducing mechanical allodynia and could be novel strategy for disease and non-opioid chronic and neuropathic pain management.

The disclosure provides for the following example embodiments, the numbering of which is not to be construed as designating levels of importance:

Embodiment 1 relates to a compound of the formula (I):

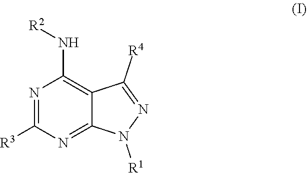

or pharmaceutically acceptable salt thereof,
wherein:
R¹ is aryl or heterocyclyl;
R² is alkyl or cycloalkyl, wherein the cycloalkyl is not amino- or amido-substituted when R¹ is aryl;
R³ and R⁴ are each interpedently H or alkyl; and
the compound is not a compound of the formula:

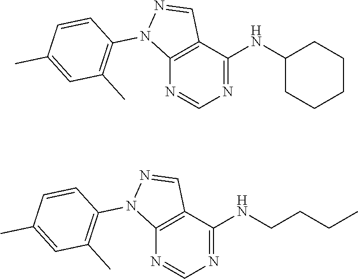 [SMILES: Cc1ccc(-n2ncc3c(NC4CCCCC4)ncnc32)c(C)c1 ; CCCCNc1ncnc2c1cnn2-c1ccc(C)cc1C]

Embodiment 2 relates to the compound of Embodiment 1, wherein R¹ is heterocyclyl.

Embodiment 3 relates to the compound of Embodiment 1 or 2, wherein R¹ is a four-, five- or six-membered heterocyclyl group.

Embodiment 4 relates to the compound of Embodiment 3, wherein the heterocyclyl group is selected from the group consisting of azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl or piperazinyl.

Embodiment 5 relates to the compound of Embodiments 1-4, wherein R³ or R⁴ is H.

Embodiment 6 relates to the compound of Embodiments 1-4, wherein R³ or R⁴ is alkyl.

Embodiment 7 relates to the compound of Embodiment 1, wherein R¹ is aryl substituted with alkyl or cycloalkyl.

Embodiment 8 relates to the compound of Embodiment claim 7, wherein alkyl is (C₁-C₆)-alkyl.

Embodiment 9 relates to the compound of Embodiment 7, wherein R¹ is not substituted with two CH₃ groups.

Embodiment 10 relates to the compound of Embodiment 7, wherein R¹ is not substituted with two CH₃ groups that are meta to one another.

Embodiment 11 relates to the compound of Embodiments 1-10, wherein R² is (C₁-C₁₀)-alkyl or (C₃-C₆)-cycloalkyl.

Embodiment 12 relates to the compound of Embodiments 1-11, wherein R² is (C₁-C₃)-alkyl, (C₅-C₁₀)-cycloalkyl or (C₃-C₅)-cycloalkyl.

Embodiment 13 relates to a compound of the formula (H):

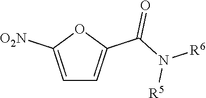

or pharmaceutically acceptable salt thereof;
wherein:
R⁵ is H, alkyl or OR⁷, wherein R⁷ is H or alkyl; and
R⁶ is aryl, rnonocyclic pyrrolidinyl or pyrrolyl; bicyclic furany; thiophenyl; indolyl, and benzimidazolyl;
wherein R⁵ is alkyl or OR⁷ when R⁵ is aryl, thiopehnyl, indolyl or benzimidazolyl.

Embodiment 14relates to the compound of Embodiment 13, wherein R⁶ is a four-, five- or six-membered heterocyclyl group.

Embodiment 15 relates to the compound of Embodiment 13 or 14, wherein the heterocyclyl group is selected from the group consisting of azetidinyl, tetrahydrofuranyl, (uranyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl or piperazinyl.

Embodiment 16 relates to the compound of Embodiment 13, wherein R⁶ is aryl.

Embodiment 17 relates to a pharmaceutical composition comprising one or more compounds of claims 1-16 and one or more pharmaceutically acceptable excipients.

Embodiment 18 relates to a method for reducing at least one of NF-κB activity and chemokine secretion induced by immunologic stimuli, the method comprising administering at least one of a 1H-pyrazolo[3,4-d]pyrimidin-4-amine compound and an 1-nitro-5-amido-disubstituted furan, or a pharmaceutically acceptable salt thereof, to a subject in need thereof.

Embodiment 19 relates to the method of Embodiment 18, further comprising administering a glucocorticosteroid or a pharmaceutically acceptable salt thereof.

Embodiment 20 relates to the method of Embodiment 18 or 19, wherein the 1H-pyrazolo[3,4-d] pyrimidin-4-amine compound acts synergistically with the glucocorticosteroid, causing a dose-sparing effect with regard to the glucocorticosteroid.

Embodiment 21 relates to the method of Embodiments 18-20, wherein the at least one 1 H-pyrazolo[3,4-d] pyri3nidin-4-amine is a compound of the formula:

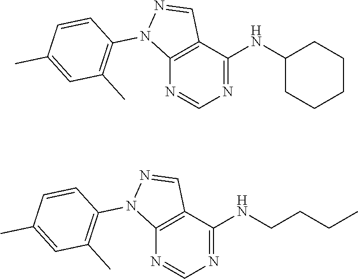 [SMILES: Cc1ccc(-n2ncc3c(NC4CCCCC4)ncnc32)c(C)c1 ; CCCCNc1ncnc2c1cnn2-c1ccc(C)cc1C]

a pharmaceutically acceptable salt thereof.

Embodiment 22 relates to the method of Embodiments 18-20, wherein the 11-1-pyrazolo[3,4-d] pyrimidin-4-amine is a compound of the formula (I):

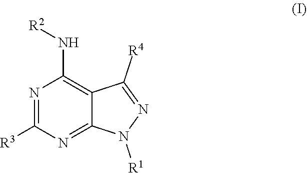

or pharmaceutically acceptable salt thereof,
wherein:
R¹ is aryl or heterocyclyl; and
R² is alkyl or cycloalkyl.

Embodiment 23 relates to the method of Embodiment 22, wherein R¹ is heterocyclyl.

Embodiment 24 relates to the method of Embodiment 22 or 23, wherein R¹ is a four-, five- or six-membered heterocyclyl group.

Embodiment 25 relates to the method of Embodiment 24, wherein the heterocyclyl group is selected from the group consisting of azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl or piperazinyl.

Embodiment 26 relates to the method of Embodiments 22-25, wherein R³ or R⁴ is H.

Embodiment 27 relates to the method of Embodiments 22-25, wherein R³ or R⁴ is alkyl.

Embodiment 28 relates to the method of Embodiment 22, wherein R¹ is aryl optionally substituted with alkyl or cycloalkyl.

Embodiment 29 relates to the method of Embodiment 28, wherein alkyl is (C₁-C₆)-alkyl.

Embodiment 30 relates to the method of Embodiments 22-29, wherein R² is (C₁-C₁₀)-alkyl or (C₃-C₆)-cycloalkyl.

Embodiment 31 relates to the method of Embodiments 22-30, wherein R² is (C₁-C₃)-alkyl, (C₅-C₁₀)-cycloalkyl or (C₃-C₅)-cycloalkyL

Embodiment 32 relates to the method of Embodiment 18, wherein the 1-nitro-5-amido-disubstituted furan is a compound of the formula (II):

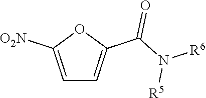

or pharmaceutically acceptable salt thereof,
wherein:
R⁵ is H, alkyl or OR⁷, wherein R⁷ is H or alkyl; and
R⁶ is aryl or heterocyclyl.

Embodiment 33 relates to the method of Embodiment 32, wherein R⁶ is a four-, five- or six-membered heterocyclyl group.

Embodiment 34 relates to the method of Embodiment 32 or 33, wherein the heterocyclyl group is selected from the group consisting of azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl or piperazinyl.

Embodiment 35 relates to the method of Embodiment 32, wherein R⁶ is aryl.

Embodiment 36 relates to a method for treating an inflammatory disease or an autoimmune disease comprising administering a therapeutically effective amount of at least one of a 1H-pyrazolo[3,4-d]pyrimidin-4-amine compound and a 1-nitro-5-amido-disubstituted furan, or a pharmaceutically acceptable salt thereof, to a subject in need thereof.

Embodiment 37 relates to a method for treating nociceptive pain or allodynia comprising administering a therapeutically effective amount of a 1-nitro--amido-disubstitued furan, or a pharmaceutically acceptable salt thereof, to a subject in need thereof.

What is claimed is:

1. A compound of the formula (I): 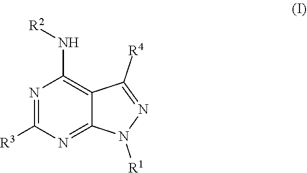
or pharmaceutically acceptable salt thereof,
wherein:
R¹ is aryl or heterocyclyl;
R² is alkyl or cycloalkyl, wherein the cycloalkyl is not amino- or amido-substituted when R¹ is aryl;
R³ and R⁴ are each independently H or alkyl; and
the compound is not a compound of the formula:

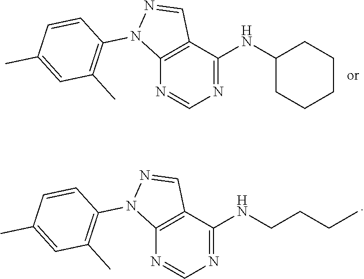 [SMILES: Cc1ccc(-n2ncc3c(NC4CCCCC4)ncnc32)c(C)c1 ; CCCCNc1ncnc2c1cnn2-c1ccc(C)cc1C]

2. The compound of claim 1, wherein R¹ is heterocyclyl.

3-4. (canceled)

5. The compound of claims 1, wherein R³ or R⁴ is H; or R³ or R⁴ is alkyl.

6. (canceled)

7. The compound of claim 1, wherein R¹ is aryl substituted with alkyl or cycloalkyl.

8-12. (canceled)

13. A compound of the formula (II): 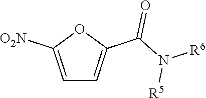
or pharmaceutically acceptable salt thereof,
wherein:
R⁵ is H, alkyl or OR⁷, wherein R⁷ is H or alkyl; and
R⁶ is aryl, monocyclic pyrrolidinyl or pyrrolyl; bicyclic furany; thiophenyl; indolyl, and benzimidazolyl;
wherein R⁵ is alkyl or OR⁷ when R⁶ is aryl, thiopehnyl, indolyl or benzimidazolyl.

14. The compound of claim 13, wherein R⁶ is a four-, five- or six-membered heterocyclyl group.

15. (canceled)

16. The method of claim 13, wherein R⁶ is aryl.

17. A pharmaceutical composition comprising one or more compounds of claim 1 and one or more pharmaceutically acceptable excipients.

18. A method for reducing at least one of NF-κB activity and chemokine secretion induced by immunologic stimuli, the method comprising administering at least one of a 1H-pyrazolo[3,4-cl]pyrimidin-4-amine compound and an 1-nitro-5-amido-disubstituted furan, or a pharmaceutically acceptable salt thereof, to a subject in need thereof.

19. The method of claim 18, further comprising administering a glucocorticosteroid or a pharmaceutically acceptable salt thereof.

20. (canceled)

21. The method of claims 18, wherein the at least one 1 H-pyrazolo[3,4-d] pyrimidin-4-amine is a compound of the formula: 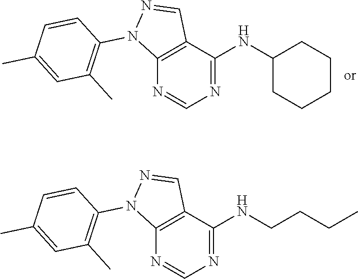 [SMILES: Cc1ccc(-n2ncc3c(NC4CCCCC4)ncnc32)c(C)c1 ; CCCCNc1ncnc2c1cnn2-c1ccc(C)cc1C]
or
a pharmaceutically acceptable salt thereof.

22. The method of claims 18, wherein the 1H-pyrazolo[3,4-d] pyrimidin-4-amine is a compound of the formula (I): 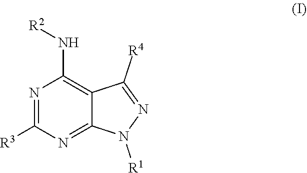
or pharmaceutically acceptable salt thereof,
wherein:
R¹ is aryl or heterocyclyl; and
R² is alkyl or cycloalkyl.

23. The method of claim 22, wherein R¹is heterocyclyl.

24-25. (canceled)

26. The method of claims 22, wherein R³ or R⁴ is H; or R³ or R³ is alkyl.

27. (canceled)

28. The method of claim 22, wherein R¹is aryl optionally substituted with alkyl or cycloalkyl.

29-31. (canceled)

32. The method of claim 18, wherein the 1-nitro-5-amido-disubstituted furan is a compound of the formula (II): 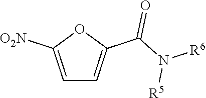
or pharmaceutically acceptable salt thereof,
wherein:
R⁵ is H, alkyl or OR⁷, wherein R⁷ is H or alkyl; and
R⁶ is aryl or heterocyclyl.

33. The method of claim 32, wherein R⁶ is a four-, five- or six-membered heterocyclyl group.

34. (canceled)

35. The method of claim 32, wherein R⁶ is aryl.

36. A method for:
(i) treating an inflammatory disease or an autoimmune disease comprising administering a therapeutically effective amount of at least one of a 1H-pyrazolo [3, 4-d]pyrimidin-4-amine compound and a 1-nitro-5-amido-disubstituted furan, or a pharmaceutically acceptable salt thereof, to a subject in need thereof; or (ii) for treating nociceptive pain or allodynia comprising administering a therapeutically effective amount of a 1-nitro-5-amido-disubstitued furan, or a pharmaceutically acceptable salt thereof, to a subject in need thereof.

37. (canceled)

38. A pharmaceutical composition comprising one or more compounds of claim 13 and one or more pharmaceutically acceptable excipients.
